(19) United States
(12) Patent Application Publication
(10) Pub. No.: US 2009/0081216 A1
(43) Pub. Date: Mar. 26, 2009

(54) TREATMENT OF PATHOLOGIES WHICH ESCAPE THE IMMUNE RESPONSE, USING OPTIMIZED ANTIBODIES

(75) Inventors: Christophe de ROMEUF, Lille (FR); Christine GAUCHER, Sequedin (FR); Sylvie JORIEUX, Villeneuve d'Ascq (FR); Dominique BOUREL, La Madelaine (FR)

Correspondence Address: FOLEY AND LARDNER LLP;SUITE 500, 3000 K STREET NW, WASHINGTON, DC, 20007, US

(73) Assignee: LABORATOIRE FRANCAIS DU FRACTIONNEMENT ET DES BIOTECHNOLOGIES

(21) Appl. No.: 12/234,609

(22) Filed: Sep. 19, 2008

Related U.S. Application Data
(63) Continuation of application No. 10/527,666, filed on Aug. 1, 2005.

(30) Foreign Application Priority Data
Sep. 13, 2002 (FR) 02/11,415
Sep. 13, 2002 (FR) 02/11,416
Jun. 12, 2003 (FR) 03/07,066

(51) Int. Cl. A61K 39/395

(52) U.S. Cl. 424/133.1

(57) ABSTRACT

The invention relates to the use of potimised human or humanized chimeric monoclonal antibodies which are produced in selected cell lines, said antibodies having a strong affvinity for receptor CD16 of the effector cells of the immune system and being able to induce the secretion of cytokines and interleukins, in particular 1“IFN? Or 1” IL2, for the treatment of pathologies for which the target cells only express a low antigenic density and in which the effector cells can only be recruited in small quantities.

The present invention relates to the use of optimized human or humanized chimeric monoclonal antibodies which are produced in selected cell lines, said antibodies having strong affinity for the CD16 receptor of the effector cells of the immune system, and also being able to induce the secretion of cytokines and of inter-leukins, in particular IFNγ or IL2, for the treatment of pathologies for which the target cells express only a low antigenic density and in which the effector cells can only be recruited in small amounts.

Immunotherapy by means of monoclonal antibodies is in the process of becoming one of the most important aspects of medicine. On the other hand, the results obtained during clinical trials appear to be contrasting. In fact, the monoclonal antibody may prove to be insufficiently effective. Many clinical trials are stopped for various reasons such as a lack of effectiveness, and side effects that are incompatible with use in clinical therapy. These two aspects are closely linked given that antibodies that are not very active are administered at high dose in order to compensate for this and to obtain a therapeutic response. The administration of high doses not only induces side effects, but it is not very economically viable.

These are major problems in the human or humanized chimeric monoclonal antibody industry.

Now, this problem is exacerbated for a certain number of pathologies for which the antigenic density expressed by the target cells is low and/or the low number of available and activated effector cells is limited, thus rendering technically impossible the use of antibodies for therapeutic purposes with the antibodies currently available. For example, in Sezary syndrome, the specific antigen, KIR3DL2, is weakly expressed (only approximately 10 000 molecules). The expression of tumor antigens may also be negatively regulated, such as HER2-neu in breast cancer. Moreover, when it is sought to inhibit angiogenesis via the targeting of VEGFR2, few molecular targets are effectively accessible since the receptor is internalized. Similarly, tumor antigen-specific peptides presented by HLA class 1 or class 2 molecules, for example in the case of carcinomas, melanomas, ovarian cancers, prostate cancers, are generally expressed very little at the surface of the target tumor cells. Finally, another situation can occur in viral infections in which the cells infected with certain viruses (HBV, HCV, HIV) express only a few viral molecules on their membrane.

This problem also arises for all pathologies which exhibit a decrease in the number of NK cells, or in their activity or in their number of CD16s (Cavalcanti M et al., Irreversible cancer cell-induced functional anergy and apoptosis in resting and activated NK cells, Int J Oncol 1999 February; 14(2): 361-6). Mention may be made, for example, of chronic myeloid leukemias (Parrado A. et al., Natural killer cytotoxicity and lymphocyte subpopulations in patients with acute leukemia, Leuk Res 1994 March; 18(3): 191-7), pathologies associated with the environment that target in particular individuals exposed to polychlorinated biphenyls (Svensson B G. et al., Parameters of immunological competence in subjects with high consumption of fish contaminated with persistent organochlorine compounds, Int Arch Occup Environ Health 1994; 65(6) 351-8), infectious diseases, in particular tuberculosis (Restrepo L M. et al., Natural killer cell activity in patients with pulmonary tuberculosis and in health controls, Tubercle 1990 June; 71(2): 95-102), chronic fatigue syndrome (CFS) (Whiteside T L, Friberg D, Natural killer cells and natural killer cell activity in chronic fatigue syndrome, Am J Med 1998 Sep. 28; 105(3A): 27S-34S), and all parasitic infections, such as, for example, schistosomula (Feldmeier H, et al., Relationship between intensity of infection and immunomodulation in human schistosomiasis. II. NK cell activity and in vitro lymphocyte proliferation, Clin Exp Immunol 1985 May; 60(2): 234-40).

Thus, the objective is to obtain novel antibodies that are more effective compared to the current antibodies, which would make it possible to envision their use in therapy for pathologies in which there are few expressed molecular targets or a low antigenic density and also a limited number of effector cells capable of being activated.

We had shown, in our application WO 01/77181 (LFB), the importance of selecting cell lines that make it possible to produce antibodies having a strong ADCC activity via FcγRIII (CD16). We had found that modifying the glycosylation of the constant fragment of the antibodies produced in rat myeloma lines such as YB2/0 resulted in the ADCC activity being improved. The glycan structures of said antibodies are of the biantennary type, with short chains, a low degree of sialylation, nonintercalated terminal attachment point mannoses and GlcNAcs, and a low degree of fucosylation.

Now, in the context of the present invention, we have discovered that the advantage of having a strong affinity for CD16 can be further enhanced by additional conditions aimed at producing antibodies which also induce the production of cytokines, in particular the production of IFNγ or IL2, by the cells of the immune system.

The abovementioned two characteristics complement one another. Specifically, the production of IFNγ or IL2 induced by the antibodies selected by means of the method of the invention can enhance the cytotoxic activity. The mechanism of action of such an activation probably stems from a positive autocrine regulation of the effector cells. It may be postulated that the antibodies bind to CD16, bringing about a cytotoxic activity, but also induce the production of IFNγ or IL2 which, in the end, results in an even greater increase in the cytotoxic activity.

We show here that the optimized antibodies of the invention maintain good effectiveness even when the antigenic density is low or the number of effector cells is limited. Thus, at doses compatible with use in clinical therapy, it is now possible to treat pathologies for which an antibody treatment could not be envisioned up until now.

### DESCRIPTION

Thus, the invention relates to the use of an optimized human or humanized chimeric monoclonal antibody, characterized in that:

a) it is produced in a cell line selected for its properties of glycosylation of the Fc fragment of an antibody, or
b) the glycan structure of the Fcgamma has been modified ex vivo, and/or
c) its primary sequence has been modified so as to increase its reactivity with respect to Fc receptors; said antibody having i) a rate of ADCC via FcγRIII (CD16) of greater than 50%, preferably greater than 100%, for an E/T (effector cell/target cell) ratio of less than 5/1, preferably less than 2/1, compared with the same antibody produced in a CHO line; and ii) a rate of production of at least one cytokine by a CD16 receptor-expressing effector cell of the immune system of greater than 50%, 100%, or preferably greater than 200%, compared with the same antibody produced in a CHO line;
for preparing a medicinal product intended for the treatment of pathologies for which the number of antigenic sites or the antigenic density is low, or the antigens are relatively inaccessible to antibodies, or else for which the number of activated or recruited effector cells is low.

Advantageously, the number of antigenic sites is less than 250 000, preferably less than 100 000 or 50 000 per target cell.

Said cytokines released by the optimized antibodies are chosen from interleukins, interferons and tissue necrosis factors (TNFs).

Thus, the antibody is selected for its ability to induce the secretion of at least one cytokine chosen from IL-1, IL-2, IL-3, IL-4, IL-5, IL-6, IL-7, IL-8, IL-9, IL-10, etc., TNFa, TGFβ, IP10 and IFNγ, by the CD16 receptor-expressing effector cells of the immune system.

Preferably, the antibody selected has the ability to induce the secretion of IFNγ or of IL2 by the CD16 receptor-expressing effector cells of the immune system, or of IL2 by Jurkat CD16 cells, for a low number of antigenic sites present at the surface of the target cells or for a low number of antigens accessible to antibodies. The amount of IFNγ or of IL2 secreted reflects the quality of the antibody bound by the CD16 receptor, as regards its antigen-binding integrity (Fc function) and effectiveness (antigenic site). In addition, the secretion of IFNγ or of IL2 by the cells of the immune system can activate the cytotoxic activity of the effector cells. Thus, the antibodies of the invention are also useful for the treatment of pathologies for which the number of activated or recruited effector cells is low.

The effector cells can express an endogenous CD16 or can be transformed. The term “transformed cell” is intended to mean a cell that has been genetically modified so that it expresses a receptor, in particular the CD16 receptor.

In a particular embodiment, the antibody of the invention is capable of inducing the secretion of at least one cytokine by a leukocytic cell, in particular of the NK (natural killer) family, or by cells of the monocyte-macrophage group. Preferably, for selecting the antibodies, a Jurkat line transfected with an expression vector encoding the CD16 receptor is used as effector cell. This line is particularly advantageous since it is immortalized and develops indefinitely in culture media. The amount of interleukin IL2 secreted reflects the quality of the antibody bound by the CD16 receptor, as regards its antigen-binding integrity (Fc function) and effectiveness (antigenic site).

In another embodiment, the optimized antibody can be prepared after having been purified and/or modified ex vivo by modification of the glycan structure of the Fc fragment. To this effect, any chemical, chromatographic or enzymatic means that is suitable for modifying the glycan structure of antibodies can be used.

In another embodiment, the antibody can be produced by cells of rat myeloma lines, in particular YB2/0 and its derivatives. Other lines can be selected for their properties of producing the antibodies defined above. Human lymphoblastoid cells, insect cells and murine myeloma cells may, for example, be tested. The selection may also be applied to the evaluation of antibodies produced by transgenic plants or transgenic mammals. To this effect, production in CHO serves as a reference (CHO being used for the production of medicinal product antibodies) for comparing and selecting the production systems producing the antibodies according to the invention.

The general glycan structure of antibodies corresponds to a biantennary type, with short chains, a low degree of sialylation, nonintercalated terminal attachment point mannoses and GlcNAcs, and a low degree of fucosylation. In these antibodies, the intermediate GlcNac content is non zero.

Thus, the invention is directed toward the use of an antibody described above, for preparing a medicinal product intended for the treatment of a pathology which escapes the immune response, in particular chosen from hemolytic disease of the newborn, Sezary syndrome, chronic myeloid leukemias, cancers in which the antigenic targets are weakly expressed, in particular breast cancer, pathologies associated with the environment that target in particular individuals exposed to polychlorinated biphenyls, infectious diseases, in particular tuberculosis, chronic fatigue syndrome (CFS), and parasitic infections such as, for example, schistosomula.

### LEGENDS AND TITLES OF THE FIGURES

FIG. 1: ADCC on red blood cells: comparison of normal red blood cells (N) versus red blood cells overexpressing the Rhesus antigen (GR6) (Teg 500 μg/well, ADCC 375 03 017).

FIG. 2: ADCC activity induced by the anti-HLA-DR chimeric antibodies expressed in CHO or YB2/0, as a function of the E/T ratio.

FIG. 3: Influence of the number of HLA-DR antigens expressed on Raji (blockade with Lym-1) on the ADCC activity induced by the anti-HLA-DR chimeric antibodies expressed in CHO (square) or YB2/0 (triangle)

FIG. 4: Influence of the number of HLA-DR antigens expressed on Raji (blockade with Lym-1) on the activation of Jurkat CD16 (IL2) induced by the anti-HLA-DR chimeric antibodies expressed in CHO (square) or YB2/0 (triangle).

FIG. 5: Influence of the number of CD20 antigens expressed on Raji (blockade with CAT 13) on the activation of Jurkat CD16.

FIG. 6: Correlation between the ADCC assay and the secretion of IL2 by Jurkat CD16.

FIG. 7: IL8 secreted by MNCs in the presence or absence of target.

FIG. 8: Secretion of cytokines by MNCs, induced by the anti-Rhesus antibodies (deduced value without target) Tox 324 03 062.

FIG. 9: Secretion of cytokines by polymorphonuclear cells, induced by the anti-Rhesus antibodies.

FIG. 10: Secretion of cytokines by NK cells, induced by the anti-Rhesus antibodies.

FIG. 11: Secretion of TNF alpha by NK cells, induced by the anti-CD20 and anti-HLA-DR antibodies expressed in CHO and YB2/0 (324 03 082).

FIG. 12: Secretion of IFN gamma by NK cells, induced by the anti-CD20 and anti-HLA-DR antibodies expressed in CHO and YB2/0 (324 03 082).

### EXAMPLE 1

### ADCC Induced by Anti-Rhesus Antibodies as a Function of the Number of Antigenic Sites

The same sequence encoding an IgG1 specific for the Rhesus D antigen is transfected into CHO and YB2/0. The cytotoxic activity of the antibodies is compared with respect to Rhesus-positive red blood cells expressing at their surface various amounts of Rhesus antigen, i.e.: normal O+ red blood cells (10-20 000 sites) and red blood cells overexpressing the Rhesus antigen (>60 000 sites).

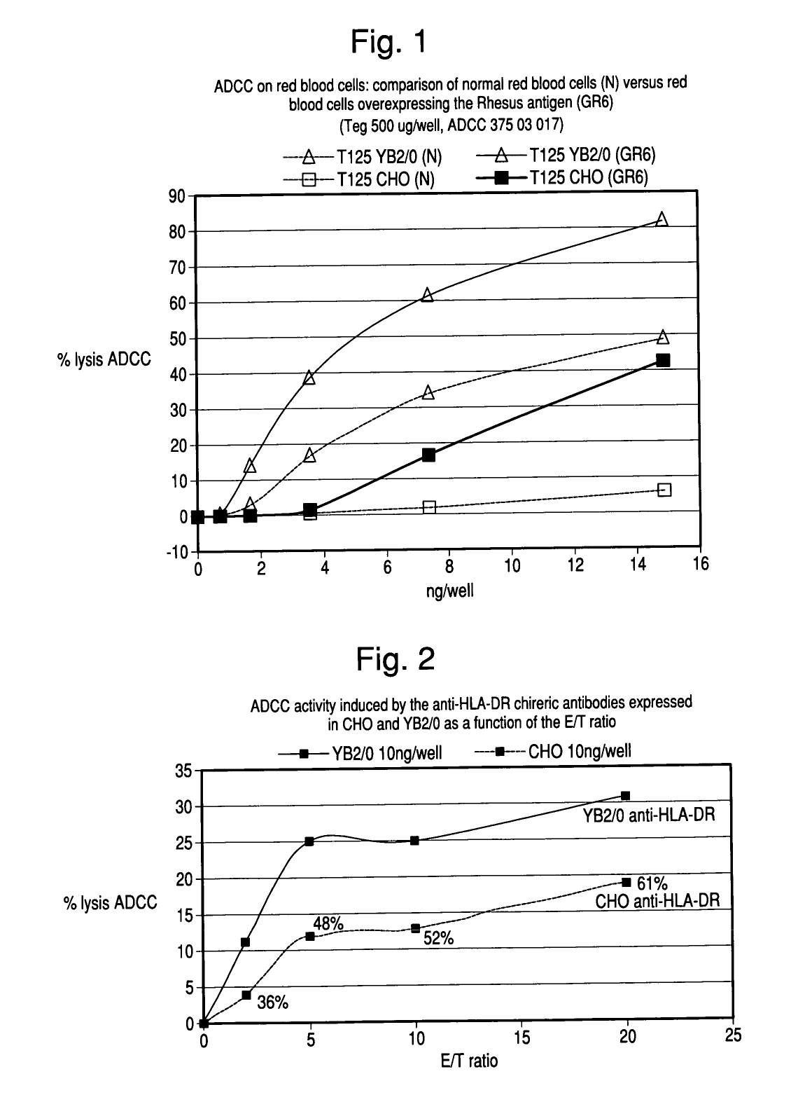

The results are given in FIG. 1:

The ADCC activity of the antibodies expressed in CHO (triangle) or YB2/0 (square) on normal red blood cells (N, open) or red blood cells overexpressing the Rhesus antigen (GR6, solid) are compared.

The difference in ADCC activity between the antibody expressed in CHO and the antibody expressed in YB2/0 is less on the red blood cells overexpressing the Rhesus antigen, especially with the high amounts of antibody, and increases as the number of antigenic sites decreases. Thus, the more the antigenic density drops, the greater the difference in ADCC activity between the antibody produced in YB2/0 and the antibody produced in CHO.

### EXAMPLE 2

### ADCC Induced by anti-HLA-DR Antibodies as a Function of the Amount of Effectors

The same sequence encoding an IgG1 specific for the HLA-DR antigen is transfected into CHO and YB2/0. The cytotoxic activity of the antibodies is compared with respect to the Raji cell in the presence of various effector/target ratios (see FIG. 2).

The difference in cytotoxic activity between the optimized antibody expressed by YB2/0 and CHO increases as the E/T ratio decreases. Thus, for the following ratios, 20/1; 10/1; 5/1; and 2/1, the relative percentage lysis induced by the antibody expressed in CHO (100% being the value of the antibody expressed in YB2/0 for each ratio) is 61%, 52%, 48% and 36%, respectively.

The antibody expressed in YB2/0 proves to be more cytotoxic than when it is produced by CHO under conditions with low amounts of effectors.

### EXAMPLE 3

### ADCC Induced by Anti-HLA-DR Antibodies as a Function of the Amount of Accessible Antigens

The same sequence encoding an IgG1 specific for the HLA-DR antigen is transfected into CHO and YB2/0. The cytotoxic activity of the antibodies is compared with respect to the Raji cell in the presence of various effector/target ratios (E/T ratio).

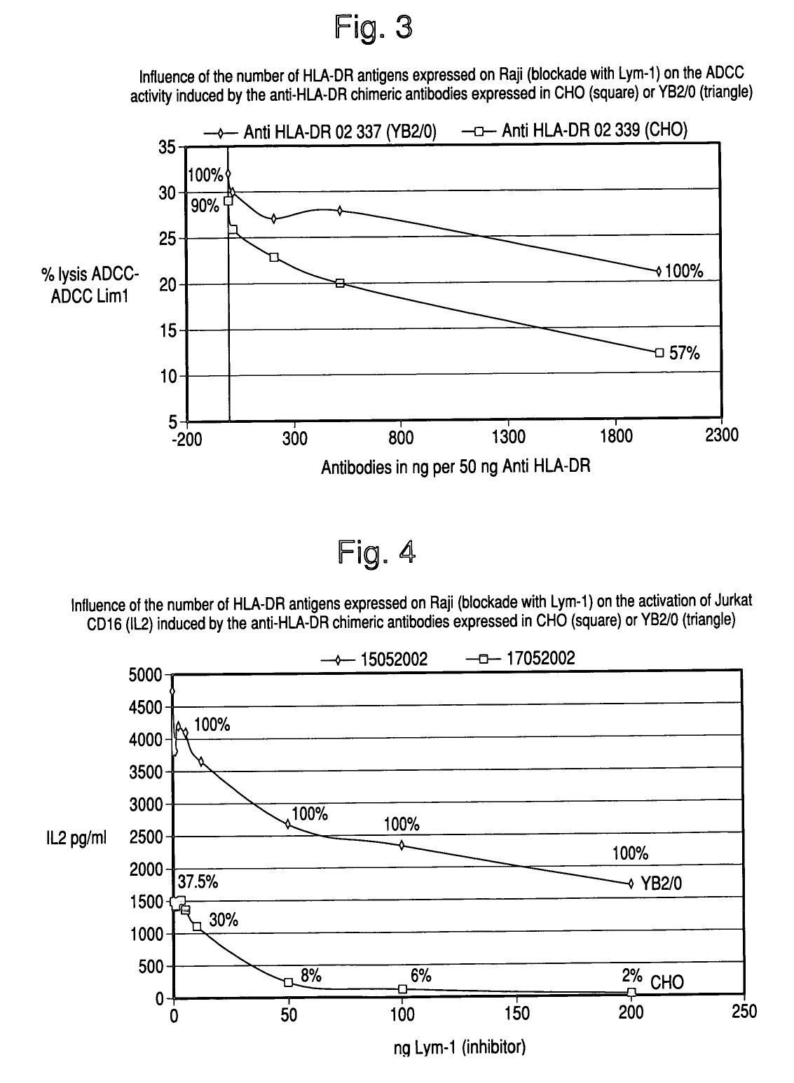

The cytotoxic activity of the antibodies is compared with respect to Raji cells for which the antigenic sites have been blocked beforehand with increasing amounts of an inactive (non-cytotoxic) anti-HLA-DR murine antibody, so as to have a decreasing number of HLA-DR antigens available with respect to the antibodies to be evaluated (see FIG. 3).

The fewer available antigenic sites there are, the greater the difference in cytotoxic activity between the optimized antibody produced in YB2/0 and the antibody produced in CHO. This indicates that one of the applications of the optimized antibody may concern target cells expressing at their surface a weakly expressed antigen recognized by the therapeutic antibody. This provides a clear therapeutic advantage compared with an antibody expressed in a CHO-type cell.

### EXAMPLE 4

### Production of IL2 by Jurkat CD16, Induced by Anti-HLA-DR Antibodies, as a Function of the Amount of Accessible Antigens

The same sequence encoding an IgG1 specific for the HLA-DR antigen is transfected into CHO and YB2/0. The activation of the effector cell (secretion of IL2 by Jurkat CD16) induced by the antibodies is compared with respect to Raji cells for which the antigenic sites have been blocked beforehand with increasing amounts of a murine anti-HLA-DR antibody, so as to have a decreasing number of HLA-DR antigens available with respect to the antibodies to be evaluated (see FIG. 4).

These results also show that the fewer available antigenic sites there are, the greater the difference in activation of the effector cells between the optimized antibody produced by YB2/0 and the antibody produced in CHO.

### EXAMPLE 5

### ADCC Induced by Anti-CD20 Antibodies as a Function of the Amount of Antigens

The results obtained with the anti-CD20 in ADCC confirm those obtained with the anti-HLADR, i.e. the lower the number of antigenic sites that are available and expressed at the surface of the target cells, the greater the difference in activation of the effector cells between the optimized antibody produced by YB2/0 and the antibody produced in CHO.

### EXAMPLE 6

### Production of IL2 by Jurkat CD16, Induced by Anti-CD20 Antibodies, as a Function of the Amount of Accessible Antigens

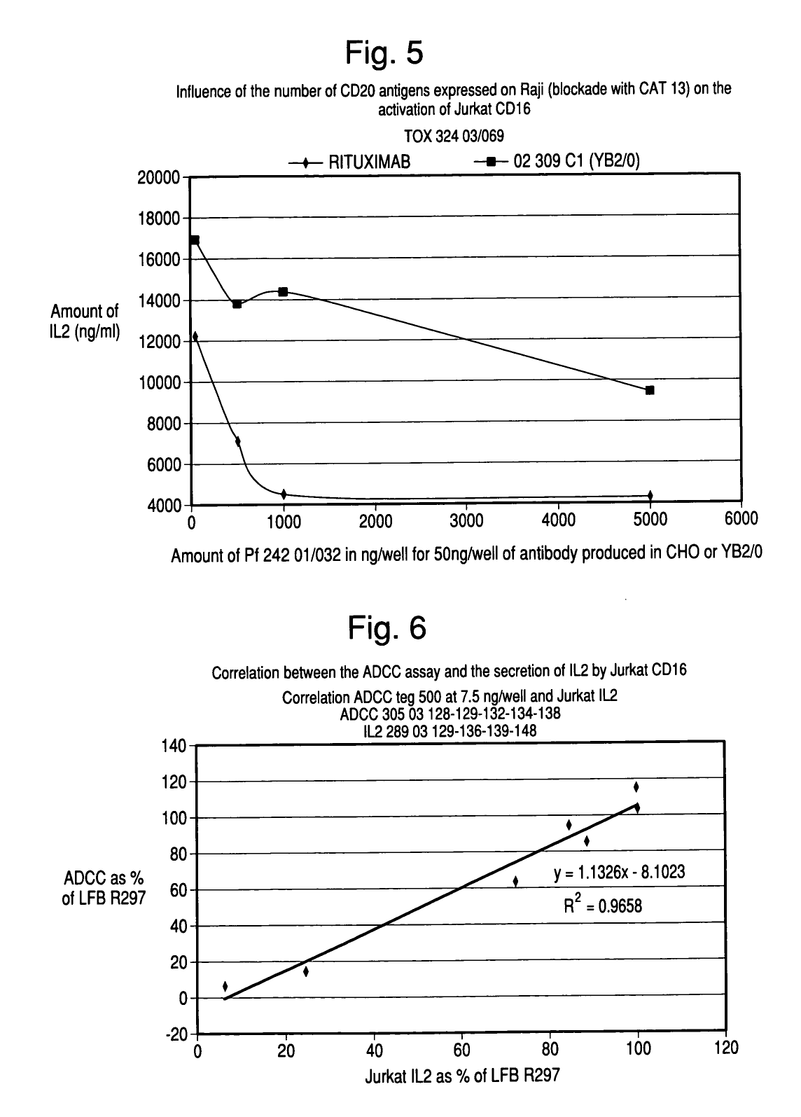

The same sequence encoding an IgG1 specific for the CD20 antigen is transfected into CHO and YB2/0. The activation of the effector cell (secretion of IL2 by Jurkat CD16), induced by the antibodies, is compared with respect to Raji cells for which the antigenic sites have been blocked beforehand with increasing amounts of an inactive murine anti-CD20 antibody, so as to have a decreasing number of CD20 antigens available with respect to the antibodies to be evaluated (see FIG. 5).

The fewer available antigenic sites there are, the greater the difference in activation of the Jurkat CD16 cells, induced by the optimized antibody produced by YB2/0 and the antibody produced in CHO. This means that a cell expressing a low antigenic density can nevertheless induce the activation of an effector cell via an optimized antibody. This capacity is much more restricted, or even zero, with an antibody expressed in CHO.

The therapeutic applications of the optimized antibody, i.e. the antibody produced in YB2/0, may thus relate to target cells expressing at their surface a weakly expressed antigen.

In conclusion, the optimized antibodies prove to be particularly useful for therapeutic applications when the target cells express few antigens at their surface, whatever the antigen.

### EXAMPLE 7

### In Vitro Correlation Between ADCC and Release of IL-2 by Jurkat CD16 Cells

For this study, 3 anti-D monoclonal antibodies were compared.

The monoclonal antibody (Mab) DF5-EBV was produced by human B lymphocytes obtained from a D-negative immunized donor and immortalized by transformation with EBV. This antibody was used as a negative control given that, in a clinical trial, it was shown to be incapable of eliminating Rhesus-positive red blood cells from the circulation.

The monoclonal antibody (Mab) DF5-YB2/0 was obtained by expressing the primary sequence of DF5-EBV in the YB2/0 line. The monoclonal antibody R297 and other recombinant antibodies were also expressed in YB2/0.

The antibodies were assayed in vitro for their ability to induce lysis of papain-treated red blood cells using mononuclear cells (PBLs) as effector.

All the assays were carried out in the presence of human immunoglobulins (IVIgs) so as to reconstitute the physiological conditions.

It is thought that IVIgs bind with high affinity to FcgammaRI (CD64). The two Mabs DF5-YB2/0 and R297 induce red blood cell lysis at a level comparable to that of the WinRho polyclonal antibodies. On the other hand, the Mab DF5-EBV is completely ineffective.

In a second series of experiments, purified NK cells and untreated red blood cells were used as effectors and targets, respectively. After incubation for 5 hours, the anti-D Mabs R297 and DF5-YB2/0 were shown to be capable of causing red blood cell lysis, whereas DF5-EBV remained ineffective.

In these two experiments, the red blood cell lysis was inhibited by the Mab 3G8 directed against FcgammaRIII (CD16).

In summary, these results demonstrate that the ADCC brought about by the Mab R297 and the Mab DF5-YB2/0 involved FcgammaRIII expressed at the surface of NK cells.

In the context of the invention, a third series of experiments was carried out using an in vitro assay with Jurkat CD16 cells in order to evaluate the effectiveness of anti-D antibodies. The Mabs were incubated overnight with Rhesus-positive red blood cells and Jurkat CD16 cells. The release of IL-2 into the supernatants was evaluated by ELISA.

A strong correlation between ADCC and activation of the Jurkat cells (production of IL2) was observed, which implies that this assay can be used to discriminate between the anti-D Mabs as a function of their reactivity toward FcgammaRIII (CD16).

The same samples are evaluated by ADCC and in the Jurkat IL2 assay. The results are expressed as a percentage relative to the “anti-D R297” reference antibody. The curve for correlation between the 2 techniques has a coefficient r2 of 0.9658 (FIG. 6).

In conclusion, these data show the importance of the post-translational modifications of the structure of antibodies and their impact on the FcgammaRIII (CD16)-specific ADCC activity. The release of cytokines such as IL-2 by the Jurkat CD16 cells reflects this activity.

### EXAMPLE 8

### Activation of NK Cells and Production of IL2 and of IFNγ

Set-up model: Jurkat cell line transfected with the gene encoding the CD16 receptor. Applications: Enhancement of an anti-tumor response. IL2, produced by the effector cells activated by antigen-antibody immunocomplexes, induces activation of T lymphocytes and of NK cells which can go as far as stimulation of cell proliferation. The IFNγ stimulates the activity of CTLs and can enhance the activity of macrophages.

### EXAMPLE 9

### Activation of Monocyte-Macrophages and Production of TNF and of IL-1Ra

Applications: Enhancement of phagocytosis and induction of anti-inflammatory properties. The TNF, produced by the effector cells activated by antigen-antibody immunocomplexes, stimulate the proliferation of tumor-infiltrating lymphocytes and macrophages. IL-1Ra is a cytokine which competes with IL1 for its receptor and thus exerts an anti-inflammatory effect.

### EXAMPLE 10

### Activation of Dendritic Cells and Production of IL10

Applications: Induction of tolerance specific to certain antigens. IL10 is a molecule that inhibits the activation of various effector cells and the production of cytokines. Thus, the IL10 produced by the effector cells activated by antigen-antibody immunocomplexes can have a regulatory role on the cytotoxic activity of the antibodies with respect to cells that are normal but express antigens that are common with the intended target cells, and can also modulate the effects of TNF alpha.

### EXAMPLE 11

### Induction of Cytokine Secretion by Various Effector Cells

Three cell populations were studied: polymorphonuclear cells, mononuclear cells and NK cells. The antibody-induction of cytokine synthesis is dependent on the presence of the target. There is little difference in the ability of the anti-D antibody R297 and of the polyclonal antibody to induce the production of various cytokines. On the other hand, AD1 very commonly does not induce cytokine secretion.

### Results:

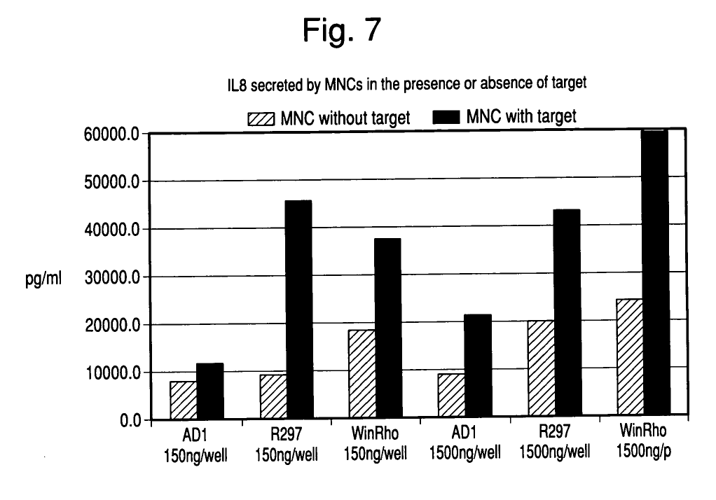

11.1 The monoclonal antibody R297 and the polyclonal antibody WinRho induce considerable secretion of IL8 in the presence of mononuclear cells. This secretion is dependent on the antibody concentration and on the presence of the antigenic target, i.e. Rh-positive red blood cells. The antibody AD1 is much less capable of inducing IL8 production (FIG. 7).

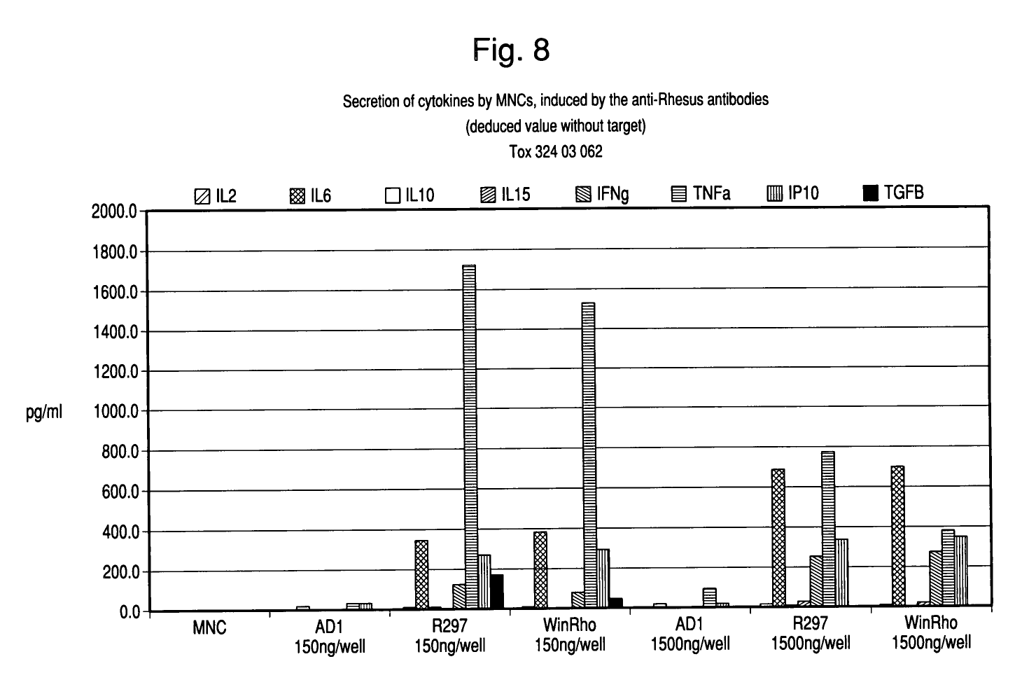

In the presence of mononuclear cells and of Rhesus-positive red blood cells, the monoclonal antibody R297 and the polyclonal anti-D antibody WinRho induce a considerable secretion of TNF alpha, and less strong, although greater than those induced by AD1, secretions of IL6, of IFN gamma, of IP10, of TNF alpha and of TGF beta. In the presence of a higher concentration of antibody, the secretion of IL6, of IFN gamma, and of IP10 increases, but that of TNF alpha and of TGF beta decreases (FIG. 8).

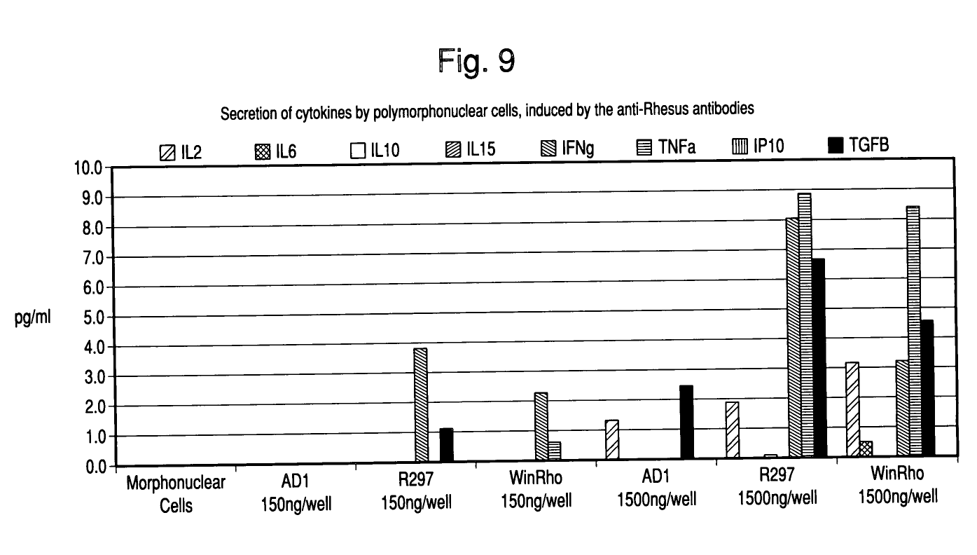

11.2 The monoclonal antibody R297 and the polyclonal anti-D antibody WinRho induce a very weak secretion, but greater than AD1, of IL2, of IFN gamma, of IP10 and of TNF by polymorphonuclear cells. This secretion is dependent on the antibody concentration (FIG. 9).

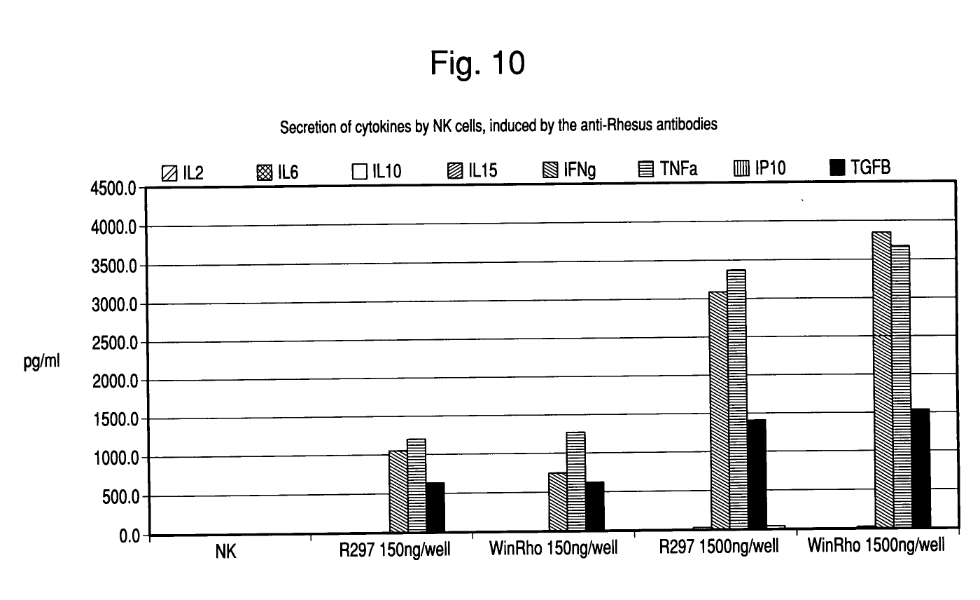

11.3 The monoclonal antibody R297 and the polyclonal anti-D antibody WinRho induce considerable secretion of IFN gamma, of IP10 and of TNF by NK cells. This secretion is dependent on the antibody concentration (FIG. 10).

### EXAMPLE 11

### Optimized Chimeric Anti-CD20 and Anti-HLA-DR Antibodies Produced in YB2/0

### Introduction

Our first results showed that the anti-D antibodies produced in YB2/0 and also the polyclonal antibodies used clinically induced the production of cytokines, in particular of TNF alpha and of interferon gamma (IFN gamma) from purified NK cells or from mononuclear cells. On the other hand, other anti-D antibodies produced in other cell lines are negative in ADCC and were found to be incapable of inducing cytokine secretion.

The additional results below show that this mechanism is not exclusive to anti-D antibodies in the presence of Rhesus-positive red blood cells, but also applies to anti-CD20 and anti-HLA-DR antibodies expressed in YB2/0. Expression in CHO cells confers on the antibody less substantial activating properties. This correlates with the results obtained in ADCC.

### Materials

### Antibodies

Anti-CD20: The anti-CD20 chimeric antibody transfected into YB2/0 is compared with a commercial anti-CD20 antibody produced in CHO (Rituxan).

Anti-HLA-DR: The same sequence encoding the anti-HLA-DR chimeric antibody is transfected into CHO (B11) or YB2/0 (4B7).

Target cells: Raji cells expressing at their surface the CD20 and HLA-DR antigen.
Effector cells: Human NK cells purified by negative selection from a human blood bag.

### Method

Various concentrations of anti-CD20 or anti-HLA-DR antibodies are incubated with the Raji cells and the NK cells. After incubation for 16 hours, the cells are centrifuged. The supernatants are assayed for TNF alpha and for IFN gamma.

### Results:

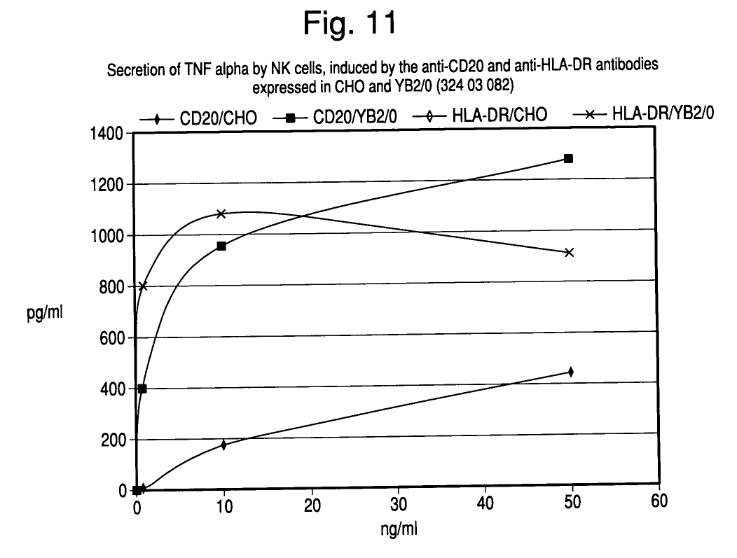

1) TNF alpha: The results are expressed in pg/ml of TNF alpha assayed in the supernatants. The various concentrations of antibodies added to the reaction mixture are given along the X-axis (FIG. 11).

The chimeric anti-CD20 and anti-HLA-DR antibodies produced in YB2/0 induce high levels of TNF in the presence of their target (Raji) compared with the same antibodies produced in CHO. The amount of TNF alpha is clearly dose-dependent on the concentration of antibody added. At 10 ng/ml of antibody, 5 times more TNF alpha is induced with the antibodies produced in YB2/0 compared with the antibodies produced in CHO.

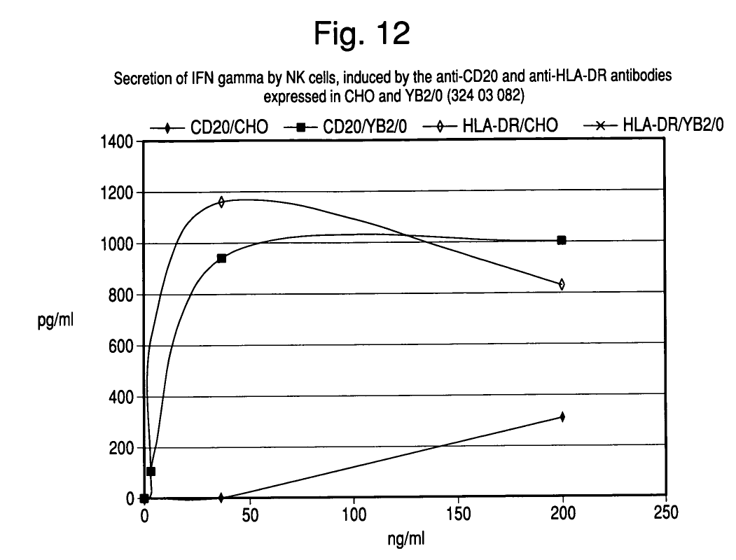

2) IFN gamma: The results are expressed in pg/ml of IFN gamma assayed in the supernatants. The various concentrations of antibodies added to the reaction mixture are given along the X-axis (FIG. 12).

The chimeric anti-CD20 and anti-HLA-DR antibodies produced in YB2/0 induce high levels of IFN gamma in the presence of their target (Raji) compared with the same antibodies produced in CHO. The amount of IFN gamma is clearly dose-dependent on the concentration of antibody added. At all the concentrations used (10 to 200 ng/ml), the anti-HLA-DR antibody produced in CHO does not induce any secretion of IFN gamma, whereas 40 ng/ml of the antibody produced in YB2/0 induces approximately 1000 pg/ml of IFN gamma.

For the anti-CD20 antibody, less than 10 ng/ml of the antibody produced in YB2/0, and 200 ng/ml of the antibody produced in CHO, are required to induce 300 pg/ml of IFN gamma (FIG. 12).

1-12. (canceled)

13. A method for treating chronic myeloid leukemia, comprising administering a human or humanized anti-HLA-DR monoclonal antibody, wherein said antibody as a general glycan structure is biantennary, with short chains, a low degree of syalylation, non-intercalated terminal attachment point mannoses and GlcNAcs, and a low degree of fucosylation and wherein the antibody is produced by rat myeloma YB2/0.
